(19) United States
(12) Patent Application Publication
(10) Pub. No.: US 2004/0248841 A1
(43) Pub. Date: Dec. 9, 2004

(54) Poly-DNP-siRNA

(76) Inventors: Jui H. Wang, Williamsville, NY (US); Hongtao Liao, Buffalo, NY (US); Long Shen, Tonawanda, NY (US); Xiaolan Chen, North Tonawanda, NY (US)

Correspondence Address: FLIESLER MEYER, LLP, FOUR EMBARCADERO CENTER, SUITE 400, SAN FRANCISCO, CA, 94111, US

(21) Appl. No.: 10/801,118

(22) Filed: Mar. 15, 2004

Related U.S. Application Data
(60) Provisional application No. 60/454,463, filed on Mar. 13, 2003.

(51) Int. Cl. A61K 48/00; C07H 21/02

(52) U.S. Cl. 514/44; 536/23.1

(57) ABSTRACT

Embodiments of the present invention provide methods and for improving the stability or efficacy of the double stranded siRNAs by replacing either one or both of its native RNA strands with homologous RNase-resistant poly-DNP-RNA to form poly-DNP-siRNA, where DNP denotes a 2′-O-(2,4-dinitrophenyl) represented by the structure:

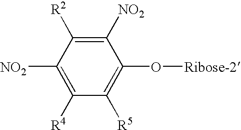

wherein R², R⁴, and R⁵ are independently H, halide, linear or branched alkyl, linear or branched acyl, linear or branched alkylene, linear or branched O-alkyl, linear or branched amido, linear or branched S-alkyl, mono or disubstituted amine, linear or branched thioamido, phosphothionate, or phosphothioate. Additional embodiments include DNP-derivatized siRNAs with improved stability or efficacy compared to non-DNP-derivatized siRNAs.

### RELATED APPLICATION

This application claims priority under 35 U.S.C. §119 to U.S. Provisional Patent Application for “Poly-DNP-siRNA”, Jui H. Wang, Hongtao Liao, Long Shen and Xiolan Chen, inventors, filed Mar. 13, 2003 using Express Mailing Label No: EJ 276 138 415 US.

### FIELD OF THE INVENTION

This invention relates generally to the field of interfering RNA.

### DESCRIPTION OF RELATED ART

Since the discovery of RNA interference (RNAi) (Fire et al., 1998; Hamilton and Baulcombe, 1999; Elbashir et al., 2001), it is generally believed that the remarkable double-stranded small interfering RNAs (siRNAs) can inhibit targeted genes at least 10-fold more effectively than the single-stranded antisense oligonucleotides. This difference in potency seems difficult to understand in view of the discovery that only the antisense strand of siRNA remains incorporated in the RNA-induced silencing complex (RISC) for guiding target cleavage in RNAi action (Martinez et al., 2002). It is possible that without hybridization the single-stranded antisense ssRNA (ssRNA) is rapidly hydrolyzed before it can be incorporated into the RISC. Chemically modified siRNA with improved stability have been synthesized (Harborth et al., 2003; Grünweller et al., 2003; Czaudema et al., 2003; Amargguioui et al., 2003) which are almost as good as but not better than the native siRNAs as specific inhibitors of gene expression.

### SUMMARY OF THE INVENTION

To overcome problems of the prior art, this invention improves small interfering double stranded siRNAs by replacing either one or both of its native RNA strands with homologous, RNase-resistant, membrane permeable poly-DNP-RNA to form poly-DNP-siRNA wherein the DNP is represented by the structure:

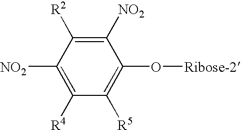

wherein R², R⁴, and R⁵ are independently H, halide, linear or branched alkyl, linear or branched acyl, linear or branched alkylene, linear or branched O-alkyl, linear or branched amido, linear or branched S-alkyl, mono or disubstituted amine, linear or branched thioamido, phosphothionate, or phosphothioate.

Preferably an oligoribonucleotide of the above structural formula has a length of between 10 and 40 nucleotides, more preferably between 12 and 30 oligoribonucleotides and more preferably still between 15 and 25 oligoribonucleotides.

### BRIEF DESCRIPTION OF THE DRAWINGS

This invention is described with reference to specific embodiments. Additional features can be appreciated by referring to the figures, in which:

FIG. 1A depicts a graph showing inhibition of A549 lung adenocarcinoma cell growth by native single and double stranded RIα RNAs. The cells were plated at a concentration of 2×10⁴/well, then treated with different concentration of double stranded RI_{α} RNAs in the presence of OLIGOFECTAMINE™. After incubation for 1 day, the liquid was removed and replaced by fresh medium without double-stranded RNA and OLIGOFECTAMINE™. After 7 more days of incubation, the cells were collected and counted with a Coulter counter. Data are expressed as the percentage of growth inhibition in reference to the growth of untreated control cells. Data are presented as mean±SD of four independent determinations.

FIG. 1B shows similar data on the inhibition of growth of A549 cells by growth inhibition poly-DNP-ssRNAs.

FIG. 2 shows the effect of chemical modification on the efficacy of siRNA for inhibiting cancer cell growth. (A) Inhibition of lung adenocarcinoma A549 cells by native siRNA and by poly-DNP-siRNA targeted at PKA/RIα. (B) Inhibition of breast cancer MCF-7 cells by siRNA and by poly-DNP-siRNA targeted at Bcl-2(α).

FIG. 3 depicts thermal denaturation profiles of homologous oligonucleotide/RNA duplexes with different backbones. DNA/RNA (●); MBO/RNA (Δ); RNA/RNA (▪); DNP-RNA/RNA (♦)

FIG. 4 depicts Western Blot analysis of IGFR protein expression level in T98G cells. Lane 1 is of molecular weight markers. Treatments were as follows: Lane 2, treated for 48 hours with 5 nM DNP-siRNA targeting IGFR(R2); lane 3, treated for 48 ours with 5 nM DNP-siRNA; lane 4, treated for 48 hours with I nM DNP-siRNA; lanes 5, 6 and 7, treated with 25, 5, and 1 nM native siRNA, respectively; lane 8, treated with OLIGOFECTAMINE™ only.

FIG. 5 depicts inhibition of T98G glioblastoma cell growth by double-stranded siRNAs targeting at IGFR(R2) in the absence of OLIGOFECTAMINE™. Scrambled poly-DNP-siRNA (control) (⋄), native siRNA (□), poly-DNP-siRNA (Δ).

### DESCRIPTION OF THE INVENTION

Embodiments of this invention include simpler general methods to improve the stability and efficacy of the double stranded siRNAs by replacing either one or both of its native RNA strands with homologous RNase-resistant, membrane-permeable poly-DNP-RNA to form poly-DNP-siRNA, where DNP denotes 2′-O-(2,4-dinitrophenyl), which may have one or more constituents other then hydrogen attached at one or more of the positions of the phenyl group as set forth by the structure as set forth above. Both siRNA and poly-DNP-siRNA were found to inhibit the growth of targeted human cancer cells in culture in a sequence-specific and concentration-dependent way, but poly-DNP-siRNAs have lower IC₅₀ values and longer lasting inhibitory effect than the corresponding unmodified siRNAs. Since poly-DNP-siRNA of any base sequence can be synthesized readily from native rNTPs and promoter-template DNA via in vitro transcription followed by a one-step derivitization reaction, these observations suggest an inexpensive general method for improving the stability and efficacy of siRNAs.

Materials and Methods

The experimental description hereinbelow utilized DNP wherein R², R⁴, and R⁵ were hydrogen. Additional descriptions of the manufacture and use of DNP-derivatized RNAs are found in U.S. Pat. Nos.: 5,496,546, 5,858,988 6,291,438, each of the above patents incorporated herein fully by reference.

Cell Culture

The cell lines used in the experiment were purchased from ATCC (Rockville, Md.). All cell culture medium and fetal bovine serum (FBS) were purchased from GIBCO-BRL (Grand Island, N.Y.). MCF-7 human breast cancer cells were grown in Minimum Essential Medium (MEM) α Medium supplemented with 5 mg/ml Insulin (Sigma, St. Louis, Mo.). A549 human lung adenocarcinoma cells were grown in F-12K nutrient Mixture, Kaighn's Modification. T98G human glioblastoma were grown in MEMα Medium. Cells grown in the corresponding medium supplemented with 10% FBS then were incubated in a humidified atmosphere of 95% air and 5% CO₂ at 37° C.

Synthesis and Derivatization of ssRNA

The ssRNA was synthesized through in vitro transcription as described before. After synthesis, the RNA was dissolved in 1 ml DEPC water and mixed with 450 μl buffer solution (2 M KHCO₃, 0.1 M K₂CO₃, pH 8.8). Then 1 ml acetone containing 160 μl of 1-fluoro-2, 4-dinitrobenzene (FDNB, Sigma) was added to the reaction mixtures. The reaction was incubated at 45° C. for 5-6 hour, the pH value was maintained around 8.8. After that, the reaction was extracted with phenol/chloroform (Sigma). The reaction mixture was dialyzed against water for 3 days to remove excess reaction reagents and salts. The ratio of DNP/RNA and the actual concentration of poly-DNP-RNA were calculated from the observed absorbance at 260 and 330 nm because the oligonucleotide has absorbance only at 260 nm, whereas the DNP has absorbance at both wavelengths.

Syntheses of siRNA and Poly-DNP-siRNA

Each single-stranded sense and antisense oligo RNA was separately collected and diluted to a desired concentration then combined together in annealing buffer (Ambion, Inc. Austin, Tex.). The mixture was heated at 90° C. in a preheated thermal cycler, followed by incubation for 1 hr at 37° C. The hybridization was checked by gel electrophoresis. The double-stranded RNA then was stored at −20° C. until ready to use.

Delivery and Cell Growth Assay

To increase the delivery of RNA into cells in culture, OLIGOFECTAMINE™ reagent (GIBCO) was used in the experiment. About 2×10⁴ cells were plated into 12-well-plate 1 day before the treatment. The RNA was added at a various concentrations in the presence of OLIGOFECTAMINE™ (1 μl/ml). After 1 day of incubation, the medium was removed and replaced by fresh medium in the absence of RNA and OLIGOFECTAMINE™. After 7-8 days of incubation, the cells were harvested and counted with a Coulter Counter (Beckman Coulter, Inc., Fullerton, Calif.) and double-checked with Trypan Blue staining. All samples were run in quadruplicate.

In order to evaluate the role of DNP groups in improving the membrane permeability of poly-DNP-siRNAs, inhibition of cell growth measurements were also made in the absence of OLIGOFECTAMINE™ under otherwise identical conditions.

Thermal Denaturation

Thermal denaturation assays of antisense oligoribonucleotides paired with complementary sense RNA strands were performed on a Hewlett Packard 8452A Diode Array Spectrophotometer equipped with temperature control. In these denaturation studies, the antisense oligoribonucleotide and its complementary sense RNA strand were each present at a concentration of 3 μM in annealing buffer (Ambion, Austin, Tex.). Each solution containing the antisense and sense strands was heated to 94° C. for 4 minutes, then slowly cooled to 37° C. and incubated for 1 hour to ensure complete pairing. The temperature was then increased while the absorbance was recorded at 260 nm.

Protein Extraction and Western Blotting

The procedure used was similar to that reported previously for studying the inhibition of MCF-7 cells by antisense RIa poly-DNP-ssRNA (Shen et al, 2003). For inhibiting IGFR (Insulin-like Growth Factor Receptor) gene expression in T98G cells, 100 nM of the specific siRNA or poly-DNP-siRNA was used. After incubation of the cells with the inhibitor in the presence of OLIGOFECTAMINE™ for 24 hours, the cells were washed once with PBS and lysed with 200 μl boiling lysis buffer (1% SOS, 1.0 mM sodium orthovanadate, 10 mM Tris, pH 7.4). Protein concentrations were determined using the BCA Protein Assay Reagent Kit (Pierce, Rockford Ill.). For Western blotting analysis each cell extract sample (30 μL loading buffer containing about 150,000 cells) was run on NuPAGE® 7 % Bis-Tris Gel with morpholine propanesulfonic acid (MOPS) running buffer, and subsequently transferred to Invitrolon™ PVDF Membranes (Invitrogen, Carlsbad, Calif.). The blotted membranes were developed using Western Breeze® Novex Chromogenic Western Blot Immodetection Kit (Invitrogen). Monoclonal antibody against IGFR was purchased from Upstate (Lake Placid, N.Y.)

Results

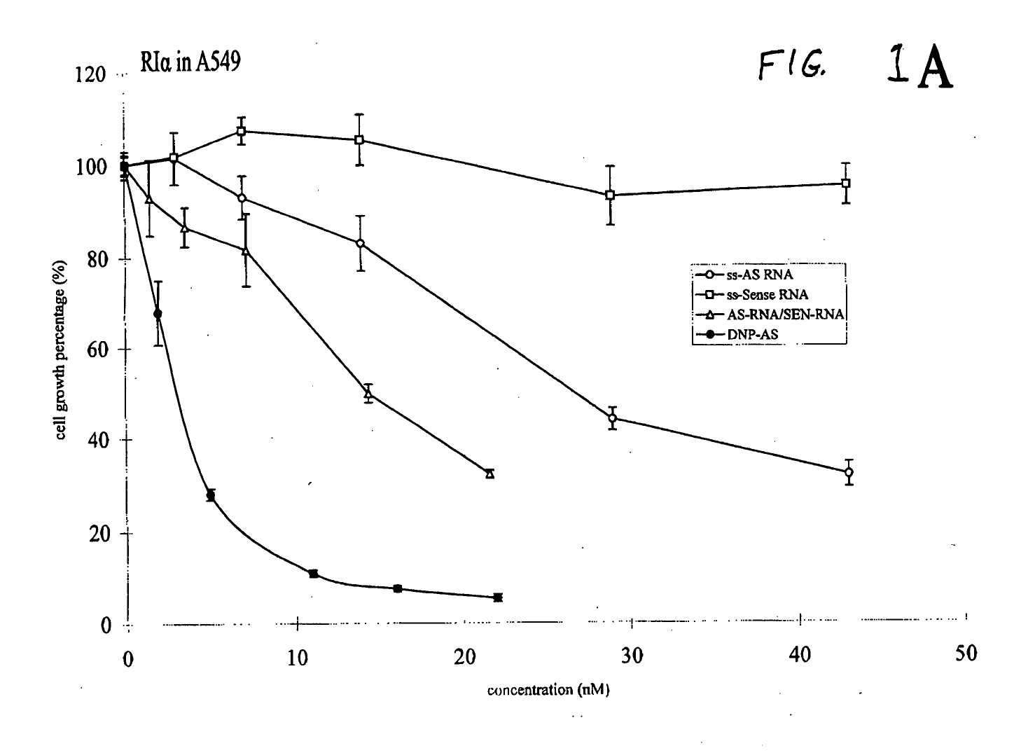

Comparative studies were carried out of inhibition of cell growth of several cancer cell lines by different native RNAs and poly-DNP-RNAs. The data on lung adenocarcinoma cells A549 with overexpressed RI_{α} gene in FIG. 1A showed that the native antisense ssRNA (AS-ssRNA) was active with IC₅₀ value of 27 nM, whereas the native duplex AS-RNA / SEN-RNA was even more active with an IC₅₀ value of 14 nM, possibly due to the resistance of the duplex to hydrolysis by endogenous RNases. FIG. 1B shows that while poly-DNP-SEN-ssRNA and the mismatched poly-DNP-MIS-RNA were both inactive, DNP-AS-ssRNA was an even more potent inhibitor (IC₅₀=3 nM) than the native duplex siRNA. While not intending to be bound by any particular theory, it is considered that this could be due to the even stronger protection of the oligoribonucleotide by DNP groups against hydrolysis by endogenous RNases (Rahman et al., 1996).

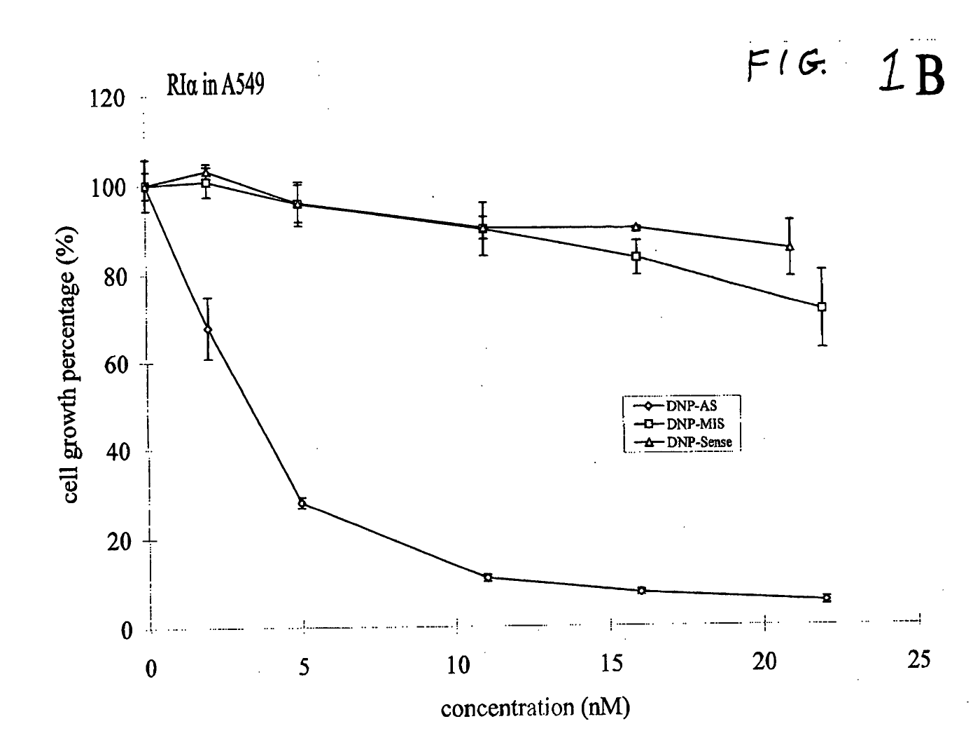

The observed concentration-dependence of the inhibition of A549 cells by the duplex siRNA and the duplexes of poly-DNP-siRNAs are illustrated in FIG. 2A. Of these four duplexes, AS-poly-DNP-RNA-SEN-poly-DNP-RNA and AS-poly-DNP-RNA-SEN-RNA were equally effective as strong inhibitors, AS-RNA-SEN-poly-DNP-RNA was a moderately strong inhibitor, but the unmodified AS-RNA-SEN-RNA (SIRNA) was the weakest inhibitor. Apparently protection of the antisense strand by the DNP groups was sufficient to give the duplex siRNA its maximum efficiency and protection of the sense strand was not necessary. This observation is consistent with the recent discovery that only the antisense strand of siRNA remains incorporated in the RNA-induced silencing complex (RISC) (Martinez et al., 2002).

Similar results were obtained on the inhibition of human breast cancer cells MCF-7 by native siRNA and by poly-DNP-siRNAs targeted at the RI_{α} gene, on the inhibition of brain tumor cells T98G by siRNA and poly-DNP-siRNAs targeted at the IGFR gene, on the inhibition of MCF-7 cells by siRNA and poly-DNP-siRNAs targeted at the Bcl-2(α) gene. FIG. 2B illustrates the MCF-7 results. The nucleotide sequences of the siRNA and poly-DNP-siRNA used in this comparative study are listed in Table I. The observed IC₅₀ values are summarized in Table II. In general, poly-DNP-siRNAs were more potent inhibitors. Since replacing only the antisense strands was sufficient to give the modified siRNA its minimal IC₅₀ value, modification of the sense strand was not needed for increased efficacy.

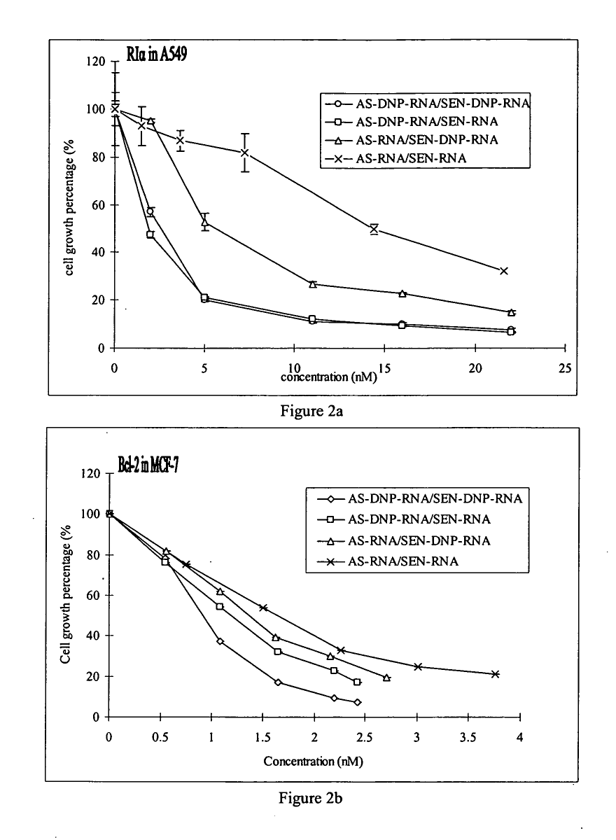

Among the four duplexes examined in FIG. 3, poly-DNP-RNA/RNA had the highest hybridization affinity. The observation that the transition temperature of poly-DNP-RNA/RNA was even higher than that of native RNA/RNA (siRNA) indicated that the DNP-groups not only did not interfere with base-pairing but may have aided hybridization via weaker interactions. This observation also suggests that during the derivatization of RNA by the present procedure, the bases themselves were not covalently labeled when the reaction was terminated at DNP/nucleoside molar ratio of 0.65 to 0.75, because otherwise the transition temperature would be appreciably lowered. These desirable features of DNP-RNA suggest that the efficacy of siRNA can be improved by DNP-derivatization.

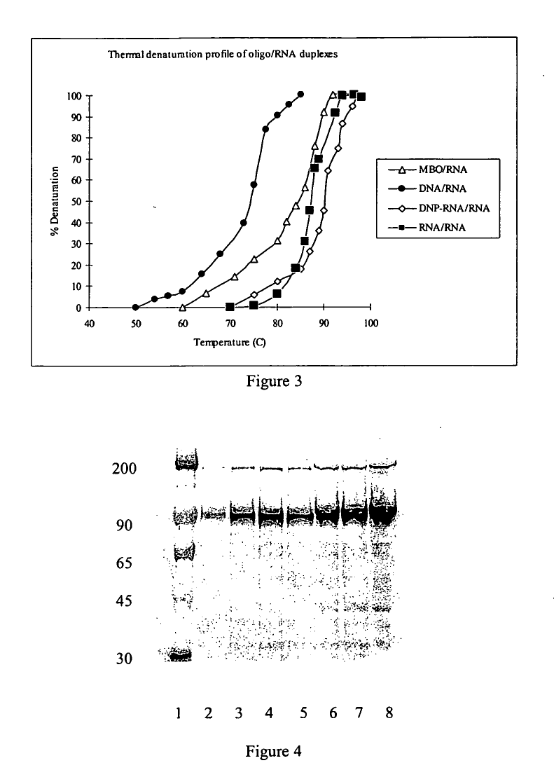

The Western Blotting data in FIG. 4 are consistent with the observed cell growth inhibition data. Although the gel was overloaded, lane 1 shows the silencing of the targeted IGFR(R2) gene by 25 nM of DNP-siRNA. The results of the Western Blotting also show that the poly-DNP-siRNA was more efficacious than the homologous native siRNA.

Antisense ssRNA and siRNA with DNP-attached backbone have two distinct advantages over other oligonucleotide inhibitors. First, poly-DNP-RNAs can be transported spontaneously into mammalian cells in the absence of transfection agents (Ashun et al., 1996; Ru et al., 1999). In the presence of transfection agents, such as OLIGOFECTAMINE™, DMRIE-C™ etc., they are transported much faster, shortening the required transport time from several days to a few hours (Cheng, 2000). Second, poly-DNP-RNAs are resistant to hydrolysis by RNases A, B, S, H, T1, T2 or phosphodiesterase I or II in solution (Rahman et al., 1996). It was also found that antisense poly-DNP-RNA can stay in live breast cancer cells for 3 or more days without losing their ability to hybridize in situ with the complementary biotin-labeled sense DNA probe (Ru et al., 1999). Oral administration of antisense poly-DNP-RNA to MLV-infected mice not only abolished viremia, but also eliminated the integrated viral genome in bone marrow (Wang and Wang, 1999). Apparently a sufficient number of intact antisense poly-DNP-RNA molecules had passed through all the membrane barriers between the gastrointestinal tract and bone marrow and reached their target in bone marrow to trigger the elimination of the MLV-infected cells.

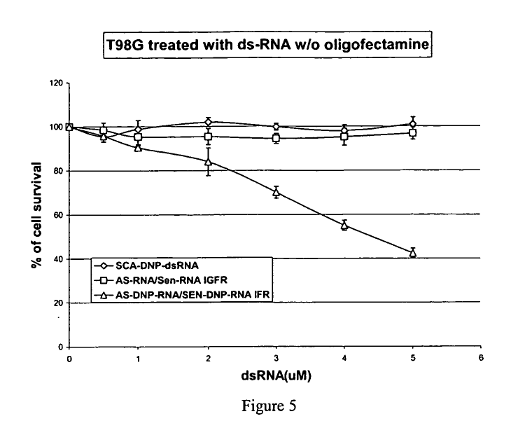

The role of DNP groups in improving membrane permeability is depicted by FIG. 5. It shows that in the absence of OLIGOFECTAMINE™ native siRNA was completely ineffective, whereas DNP-siRNA was still active with an albeit much higher IC₅₀ value.

Because of their enhanced bioavailability, poly-DNP-siRNAs have higher inhibition efficacy than the homologous native siRNAs, although the extent of improvement will vary with the membrane permeability and endogenous RNase activity of the chosen type of cells. The improved efficacy should be advantageous for both gene-silencing and therapeutic studies.

TABLE I

Sequences of siRNAs and DNP-siRNAs Tested

| | |
|---|---|
| Target | Sequence |

| | |
|---|---|
| IGFR (1) | 5′-GGGACCCUCCUCCGGAGCCAG-3′ |
| | 3′-GCCCGUGGGAGGAGGCCUCGG-5′ |
| IGFR (R2) | 5′-GCCGAUGUGUGAGAAGACCUU-3′ |
| | 3′-UUCGGCUACACACUCUUCUGG-5′ |
| Bcl-2 | 5′-GGGUGCGCCAUAUUUCCCAGA-3′ |
| | 3′-CACGCGGUAUAAAGGGUCUAG-5′ |
| RIα | 5′-GGCUGCGUGCCUCCUCACUGG-3′ |
| | 3′-GACGCACGGAGGAGUGACCGG-5′ |

TABLE II
Inhibition of growth of targeted cancer cells
IC₅₀ values of siRNA and DNP-siRNA Homologous Oligoribonucleotides

| | | siRNA | | DNP-siRNA | | |
|---|---|---|---|---|---|---|
| | | | Oligo- | | Oligo- | |
| | | IC₅₀ | fectamine | IC₅₀ | fectamine | IC₅₀ |
| Cell line | Target gene | (nM) | (μl/l) | (nM) | (μl/l) | ratio |
| MCF-7 | PKA-R1α | 0.3 | 1 | 0.15 | 1 | 2 |
| MCF-7 | Bcl-2 (α) | 1.7 | 1 | 0.9 | 1 | 2 |
| MCF-7 | IGFR (1) | 2.5 | 3 | 0.4 | 3 | 6 |
| MCF-7 | IGFR (R2) | 0.5 | 3 | 0.1 | 3 | 5 |
| A549 | PKA/R1α | 15 | 1 | 2.5 | 1 | 6 |
| A549 | IGFR (1) | 75 | 1.5 | 10 | 1.5 | 7.5 |
| A549 | IGFR (R2) | 18 | 3 | 2.5 | 3 | 7 |
| T98G | PKA/R1α | 22 | 3 | 4 | 3 | 5.5 |
| T98G | IGFR (1) | 100 | 3 | 7 | 1.5 | 14 |
| T98G | IGFR (R2) | 15 | 3 | 4 | 3 | 4 |

The disclosures of the following references are incorporated herein by reference thereto:

1. AMARZGUIOUI, M., HOLEN, T., BABLE, E., PRYDZ, H. (2003), Tolerance for mutations and chemical modifications in a siRNA. Nucleic Acid Res. 31, 589-595.

2. ASHUN, M. A., HU, Y., KANG, I., LI, C. C. and WANG, J. H. (1996). Inhibition of murine leukemia virus with poly-2′-O-(2,4-dinitrophenyl)poly[A]. Antimicrobial Agents Chemotherapy 40, 2311-2317.

3. CZAUDERNA, F., FECHTNER, M., DAMES, S., AYGUN, H., KLIPPEL, A., PRONK, G. J., GIESE, K., KAUFMANN, J. (2003). Structural variations and stabilizing modifications of synthetic siRNAs in mammalian cells. Nucleic Acids Research, 31, 2705-2716.

4. ELBASHIR, S. M., HARBOTH, J., LENDECKEL, W., YALCIN,A., WEBER, K. and TUSCHL, T. (2001) Duplexes of 21-nucleotide RNAs mediate RNA interference in mammalian cell culture. Nature, 411, 494-498.

5. FIRE, A., XU, S., MONTGOMERY, M. K., KOSTAS, S. A., DRIVER, S. E. and MELLO, C. C. (1998). Potent and specific genetic interference by double-stranded RNA in Caenorhabditis elegans. Nature 391, 806-811.

6. GRÜNWELLER, A., WYSZKO, E., BIEBER, B., JAHNEL, R., ERDMANN, V. A., KURRECK, J. (2003). Comparison of different antisense strategies in mammalian cells using locked nucleic acids 2′-O-methyl RNA, phosphorothioates and small inerfering RNA. Nucleic Acid Res. 31, 3185-3193.

7. HAMILTON, A. J. and BAULCOMBE (1999). A species of small antisense RNA in post-transcriptional gene silencing in plants. Science 286, 950-952.

8. HANNON, G. J. (2002) RNA interference. Nature 418, 244-251.

9. HARBORTH, J., ELBASHIR, S. M., VANDENBURGH, K., MANNINGA, H., SCARINGE, S. A., WEBER, K. and TUSCHL, T. (2003). Sequence, chemical, and structural variation of small interfering RNAs and short hairpin RNAs and the effect on mammalian gene silencing. Antisense Nucleic Acid Drug Dev. 13, 83-105.

10. MARTINEZ, J., PATKANOWSKA, A., URLAUB, H., LUHRMANN, R. and TUSCHL, T. (2002). Single-stranded antisense siRNAs guide target RNA cleavage in RNAi. Cell 110, 563-574.

11. RAHMAN, M. H., KANG, I., WATERBURY, R. G., NARANG, U., BRIGHT, F. V. and WANG, J. H. (1996). Selective removal of ribonucleases from solution with covalently anchored macromolecular inhibitor. Anal. Chem. 68, 134-138.

12. RU, K., SCHMITT, S., JAMES, W. I. and WANG, J. H. (1999). Growth inhibition and antimetastatic effect of antisense poly-DNP-RNA on human breast cancer cells. Oncol. Res. 11, 505-512.

13. WANG, A. AND WANG, J. H. (1999). Effective Treatment of Murine Leukemia with Antisense Poly-2′O-(2,4-Dinitrophenyl)-Oligoribonucleotides. Antisense & Nucleic Acid Drug Development 9, 43-51.

While the present invention has been described in connection with specific embodiments thereof, it will be understood that it is capable of further modification, and this application is intended to cover any variations, uses, or adaptations of the invention following, in general, the principles of the invention and including such departures from the present invention as come within known or customary practice in the art to which the invention pertains and as may be applied to the essential features hereinbefore set forth, and as fall within the scope of the invention and any equivalents thereof.

We claim:

1. A double stranded siRNA compound wherein one or both of said RNA strands are derivatized by DNP to form a poly-DNP-siRNA, where DNP denotes a 2′-O-(2,4-dinitrophenyl), wherein positions 3, 5 and 6 of said phenyl group have attached thereto R², R⁴, and R⁵, respectively, and wherein R², R⁴, and R⁵ are independently selected from the group consisting of H, halide, linear or branched alkyl, linear or branched acyl, linear or branched alkylene, linear or branched O-alkyl, linear or branched amido, linear or branched amido, linear or branched S-alkyl, mono or disubstituted amine, linear or branched thioamido, phosphothionate and phosphothioate.

2. The compound as set forth in claim 1, wherein R², R⁴, and R⁵ are hydrogen.

3. A method of increasing the stability or efficacy of a double stranded siRNA compound comprising
forming one or both of the native RNA strands as a homologous RNase-resistant RNA, to form a poly-DNP-siRNA, where DNP denotes a 2′-O-(2,4-dinitrophenyl) wherein positions 3, 5 and 6 of said-phenyl group have attached thereto R², R⁴, and R⁵, respectively, and wherein R², R⁴, and R⁵ are independently selected from the group consisting of H, halide, linear or branched alkyl, linear or branched acyl, linear or branched alkylene, linear or branched O-alkyl, linear or branched amido, linear or branched S-alkyl, mono or disubstituted amine, linear or branched thioamido, phosphothionate and phosphothioate.

4. The method as set forth in claim 3, wherein R², R⁴, and R⁵ are each hydrogen.

5. A method of silencing a targeted gene comprising introducing into a cell containing the targeted gene a poly-DNP-siRNA, where DNP denotes a 2′-O-(2,4-dinitrophenyl), wherein positions 3, 5 and 6 of said phenyl group have attached thereto R², R⁴, and R⁵, respectively, and wherein R², R⁴, and R⁵ are independently selected from the group consisting of H, halide, linear or branched alkyl, linear or branched acyl, linear or branched alkylene, linear or branched O-alkyl, linear or branched amido, linear or branched amido, linear or branched S-alkyl, mono or disubstituted amine, linear or branched thioamido, phosphothionate and phosphothioate.

6. The method as set forth in claim 5, wherein R², R⁴, and R⁵ are hydrogen.

7. In a method for administering a double-stranded siRNA compound, the improvement comprising an siRNA compound, wherein one or both of said RNA strands are derivatized by DNP to form a poly-DNP-siRNA, where DNP denotes a 2′-O-(2,4-dinitrophenyl), wherein positions 3, 5 and 6 of said phenyl group have attached thereto R², R⁴, and R⁵, respectively, and wherein R², R⁴, and R⁵ are independently selected from the group consisting of H, halide, linear or branched alkyl, linear or branched acyl, linear or branched alkylene, linear or branched O-alkyl, linear or branched amido, linear or branched S-alkyl, mono or disubstituted amine, linear or branched thioamido, phosphothionate and phosphothioate.

8. The method as set forth in claim 7, wherein R², R⁴, and R⁵ are hydrogen.

9. The improvement of claim 7, wherein the oligoribonucleotide has a length of between 10 and 40 nucleotides.

10. The improvement of claim 7, wherein the oligoribonucleotide has a length of between 12 and 30 nucleotides.

11. The improvement of claim 7, wherein the oligoribonucleotide has a length of between 15 and 25 nucleotides.

12. The improvement of claim 8, wherein the oligoribonucleotide has a length of between 10 and 40 nucleotides.

13. The improvement of claim 8, wherein the oligoribonucleotide has a length of between 12 and 30 nucleotides.

14. The improvement of claim 8, wherein the oligoribonucleotide has a length of between 15 and 25 nucleotides.

15. In a therapeutic method for down-regulating gene expression using siRNA, the improvement comprising an siRNA compound, wherein one or both of said RNA strands are derivatized by DNP to form a poly-DNP-siRNA, where DNP denotes a 2′-O-(2,4-dinitrophenyl), wherein positions 3, 5 and 6 of said phenyl group have attached thereto R², R⁴, and R⁵, respectively, and wherein R², R⁴, and R⁵ are independently selected from the group consisting of H, halide, linear or branched alkyl, linear or branched acyl, linear or branched alkylene, linear or branched O-alkyl, linear or branched amido, linear or branched S-alkyl, mono or disubstituted amine, linear or branched thioamido, phosphothionate and phosphothioate.

16. In the improvement of claim 15, wherein R², R⁴, and R⁵ are hydrogen.

17. In the improvement of claim 15, wherein the oligoribonucleotide has a length of between 10 and 40 nucleotides.

18. In the improvement of claim 15, wherein the oligoribonucleotide has a length of between 12 and 30 nucleotides.

19. In the improvement of claim 15, wherein the oligoribonucleotide has a length of between 15 and 25 nucleotides.

20. In the improvement of claim 16, wherein the oligoribonucleotide has a length of between 10 and 40 nucleotides.

21. In the improvement of claim 16, wherein the oligoribonucleotide has a length of between 12 and 30 nucleotides.

22. In the improvement of claim 16, wherein the oligoribonucleotide has a length of between 15 and 25 nucleotides.


SEQUENCE LISTING (8 sequences)

SEQ ID NO: 1 - DNA, 21 bp, Human
gggacccuccuccggagccag

SEQ ID NO: 2 - DNA, 21 bp, Human
gccccugggaggaggccucgg

SEQ ID NO: 3 - DNA, 21 bp, Human
gccgaugugugagaagaccuu

SEQ ID NO: 4 - DNA, 21 bp, Human
uucggcuacacacucuucugg

SEQ ID NO: 5 - DNA, 21 bp, Human
gcgugcgccauauuucccaga

SEQ ID NO: 6 - DNA, 21 bp, Human
cacgcgguauaaagggucuag

SEQ ID NO: 7 - DNA, 21 bp, Human
ggcugcgugccuccucacugg

SEQ ID NO: 8 - DNA, 21 bp, Human
gacgcacggaggagugaccgg